(19) United States
(12) Patent Application Publication
(10) Pub. No.: US 2012/0071536 A1
(43) Pub. Date: Mar. 22, 2012

(54) N-[(2-AZABICYCLO[2.1.1]HEX-1-YL)-ARYL-METHYL]-BENZAMIDE DERIVATIVES, PREPARATION THEREOF, AND THERAPEUTIC USE THEREOF

(75) Inventors: Gihad Dargazanli, Paris (FR); Genevieve Estenne-Bouhtou, Paris (FR); Abdel-Kader Mafroud, Paris (FR)

(73) Assignee: SANOFI, Paris (FR)

(21) Appl. No.: 13/148,583

(22) PCT Filed: Feb. 9, 2010
(86) PCT No.: PCT/FR2010/050203

§ 371 Date: Dec. 1, 2011

(30) Foreign Application Priority Data
Feb. 10, 2009 (FR) 0900578

(51) Int. Cl. A61K 31/403; A61P 25/00; A61P 29/00; A61P 25/22; A61P 25/18; A61P 25/32; A61P 25/06; C07D 209/52; A61P 25/24

(52) U.S. Cl. 514/412; 548/452

(57) ABSTRACT

The invention relates to N-[(2-aza-bicyclo[2.2.1]hex-1-yl)-aryl-methyl]-benzamide derivatives of the general formula (I) where: R is a hydrogen atom or a (C₁-C₆)alkyl group, (C₃-C₇)-cycloalkyl, optionally substituted by one or more fluorine, (C₃-C₇)-cycloalkyl, (C₂-C₄)alkenyl, phenyl, (C₁-C₆)alkoxy, hydroxy; R₁ is phenyl or naphtyl optionally substituted by one or more halogen, (C₁-C₆)alkyl, (C₁-C₆)alkoxy, halo-(C₁-C₆)alkyl, NR₄R₅, (C₁-C₆)alkylthio, (C₁-C₆)alkyl-SO₂, phenyl or heteroaryl; R₂ is one or more hydrogen or halogen atoms, halo-(C₁-C₆)alkyl, (C₁-C₆)alkyl, (C₃-C₇)cycloalkyl, (C₃-C₇)-cycloalkyl-(C₁-C₃)alkyl; R₃, R₄ and R₅ are independently a hydrogen atom or a (C₁-C₆)alkyl group; R₆ is a (C₁-C₆)alkyl group; R₃ and R₄ as well as R₃ and R₆ may form, together with the atoms having them, a 5- or 6-member ring; R₄ and R₅ may form together a ring selected from azetidine, pyrrolidine, piperidine, morpholine, thiomorpholine, piperazine, azepine, optionally substituted by a (C₁-C₆)alkyl group. The invention also relates to the therapeutic use thereof and to a method for synthesizing same.

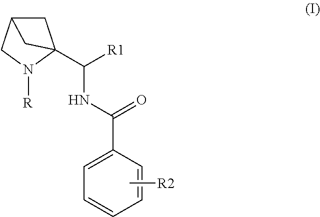

The present invention relates to N-[(2-azabicyclo[2.2.1]hex-1-yl)(aryl)methyl]-benzamide derivatives, to their preparation and to their therapeutic application in the treatment or prevention of diseases involving glycine transporters GlyT1.

The compounds of the invention correspond to the general formula (I)

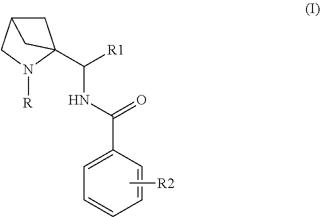

in which:
R represents a hydrogen atom or a group chosen from (C₁-C₆)alkyl or (C₃-C₇)cyclo-alkyl groups, these groups optionally being substituted by one or more groups chosen, independently of one another, from the fluorine atom or (C₃-C₇)cycloalkyl, (C₂-C₄)alkenyl, phenyl, (C₁-C₆)alkoxy or hydroxyl groups; the phenyl group is optionally substituted by one or more (C₁-C₆)alkoxy groups;
R₁ represents a phenyl or naphthyl group which is optionally substituted by one or more substituents chosen, independently of one another, from halogen atoms or (C₁-C₆)alkyl, (C₁-C₆)alkoxy, halo(C₁-C₆)alkyl, NR₄R₅, NR₃C(O)OR₄, NR₃SO₂R₄, NR₃C(O)R₆, hydroxyl, halo(C₁-C₆)alkoxy, (C₁-C₆)alkylthio, (C₁-C₆)alkyl-SO₂, phenyl or heteroaryl groups, the phenyl group being optionally substituted by one or more substituents independently chosen from halogen atoms or (C₁-C₆)alkyl, (C₁-C₆)alkoxy, halo(C₁-C₆)alkyl, NR₄R₅, NR₃C(O)OR₄, NR₃SO₂R₄, NR₃C(O)R₆, hydroxyl, halo(C₁-C₆)alkoxy, (C₁-C₆)alkylthio or (C₁-C₆)alkyl-SO₂ groups and the heteroaryl group being optionally substituted by one or more substituents independently chosen from halogen atoms or (C₁-C₆)alkyl, (C₁-C₆)alkoxy, halo(C₁-C₆)alkyl or NR₄R₅ groups;
R₂ represents one or more substituents chosen from the hydrogen atom, halogen atoms or (C₁-C₆)alkyl, (C₃-C₇)cycloalkyl, (C₃-C₇)cycloalkyl(C₁-C₃)alkyl, halo(C₁-C₆)alkyl, (C₁-C₆)alkoxy, NR₄R₅, phenyl, heteroaryl, cyano, acetyl, (C₁-C₆)alkylthio, (C₁-C₆)alkylsulphonyl, carboxyl or (C₁-C₆)alkoxycarbonyl groups; the phenyl group being optionally substituted by one or more substituents independently chosen from halogen atoms or (C₁-C₆)alkyl, (C₁-C₆)alkoxy, halo(C₁-C₆)alkyl, NR₄R₅, NR₃C(O)OR₄, NR₃SO₂R₄, NR₃C(O)R₆, hydroxyl, halo(C₁-C₆)alkoxy, (C₁-C₆)alkylthio or (C₁-C₆)alkyl-SO₂ groups and the heteroaryl group being optionally substituted by one or more substituents independently chosen from halogen atoms or (C₁-C₆)alkyl, (C₁-C₆)alkoxy, halo(C₁-C₆)alkyl or NR₄R₅ groups;
R₃, R₄ and R₅ represent, independently of one another, a hydrogen atom or a (C₁-C₆)alkyl group;
R₆ represents a (C₁-C₆)alkyl group;
R₄ and R₅ can together form, with the nitrogen atom which carries them, a ring chosen from azetidine, pyrrolidine, piperidine, morpholine, thiomorpholine, piperazine or azepine rings optionally substituted by a (C₁-C₆)alkyl group;
R₃ and R₄ can together form, with the atoms which carry them, a 5- or 6-membered ring;
R₃ and R₆ can together form, with the atoms which carry them, a 5- or 6-membered ring;
   in the form of the base or of an addition salt with an acid.

The compounds of formula (I) comprise an asymmetric carbon atom. They can thus exist in the form of enantiorners. These enantiomers, including racemic mixtures, come within the scope of the invention.

The compounds of formula (I) can exist in the form of bases or of addition salts with acids. Such addition salts come within the scope of the invention.

These salts are advantageously prepared with pharmaceutically acceptable acids but the salts of other acids, for example for use in the purification or isolation of the compounds of formula (I), also come within the invention.

In the context of the invention:
Cₜ-C_{z}, where t and z can take the values from 1 to 6, is understood to mean a carbon chain which can have from t to z carbon atoms; for example, C₁-C₆ is understood to mean a carbon chain which can have from 1 to 6 carbon atoms;
alkyl is understood to mean a saturated, linear or branched, aliphatic group; for example, a (C₁-C₆)alkyl group represents a linear or branched carbon chain of 1 to 6 carbon atoms, for example a methyl, ethyl, propyl, isopropyl, butyl, isobutyl, tert-butyl, pentyl or hexyl;
alkenyl is understood to mean a mono- or polyunsaturated and linear or branched aliphatic group comprising, for example, 1 or 2 ethylenic unsaturations,
alkylene is understood to mean a saturated, linear or branched, divalent alkyl group; for example, a C₁₋₆-alkylene group represents a linear or branched divalent carbon chain of 1 to 6 carbon atoms, for example a methylene, ethylene, 1-methyl-ethylene or propylene,
amino is understood to mean an NH₂ group;
alkoxy is understood to mean an —O-alkyl group,
acetyl is understood to mean a —C(O)— group,
cyano is understood to mean a —CN group,
hydroxyl is understood to mean an —OH group,
halogen atom is understood to mean a fluorine, a chlorine, a bromine or an iodine,
haloalkyl is understood to mean an alkyl group, one or more hydrogen atoms of which have been replaced by a halogen. Mention may be made, by way of examples, of the trifluoromethyl, trifluoroethyl or pentafluoroethyl groups,
heteroaryl is understood to mean a 5- or 6-membered aromatic monocyclic group comprising from 1 to 3 heteroatoms chosen from nitrogen, oxygen and sulphur. Mention may be made, as examples of heteroaryl group, of the pyrrole, furan, thiophene, pyrazole, imidazole, triazole, tetrazole, oxazole, isoxazole, oxadiazole, thiazole, isothiazole, thiadiazole, pyridine, pyrimidine, pyrazine, pyridazine or triazine groups.

Among the compounds of general formula (I) which are subject-matters of the invention, a first group of compounds is composed of the compounds for which R represents a hydrogen atom or a (C₁-C₆)alkyl group optionally substituted by one or more groups chosen, independently of one another, from the fluorine atom or (C₂-C₄)alkenyl, hydroxyl, (C₃-C₇)cycloalkyl or phenyl groups;

R₁, R₂, R₃, R₄, R₅ and R₆ being as defined above.

Among the compounds of general formula (I) which are subject-matters of the invention, a second group of compounds is composed of the compounds for which R represents a hydrogen atom or a methyl, ethyl, propyl, isobutyl or allyl group, the methyl, ethyl or isobutyl group or groups being optionally substituted by one or more groups chosen, independently of one another, from the fluorine atom, the hydroxyl group, a cyclopropyl group or a phenyl group;

R₁, R₂, R₃, R₄, R₅ and R₆ being as defined above.

Among the compounds of general formula (I) which are subject-matters of the invention, a third group of compounds is composed of the compounds in which R₁ represents a phenyl or naphthyl group optionally substituted by one or more halogen atoms or (C₁-C₆)-alkyl, (C₁-C₆)alkoxy, halo(C₁-C₆)alkyl, NR₄R₅ or hydroxyl groups;

R, R₂, R₃, R₄ and R₅ being as defined above.

Among the compounds of general formula (I) which are subject-matters of the invention, a fourth group of compounds is composed of the compounds in which R₁ represents a phenyl or naphthyl group optionally substituted by one or more halogen atoms or methyl, methoxy, trifluoromethyl, NH₂ or hydroxyl groups;

R, R₂, R₄ and R₅ being as defined above.

Among the compounds of general formula (I) which are subject-matters of the invention, a fifth group of compounds is composed of the compounds for which R₂ represents one or more substituents chosen from the hydrogen atom, halogen atoms or NR₄R₅, (C₁-C₆)alkoxy, halo(C₁-C₆)alkyl, (C₁-C₆)alkylthio or (C₁-C₆)alkyl-SO₂ groups;

R, R₁, R₃, R₄, R₅ and R₆ being as defined above.

Among the compounds of general formula (I) which are subject-matters of the invention, a sixth group of compounds is composed of the compounds for which R₂ represents one or more substituents chosen from the hydrogen atom, halogen atoms or methyl, ethyl, NH₂, methoxy, trifluoromethyl, methanesulphanyi or ethanesulphonyl groups;

R, R₁, R₃, R₄, R₅ and R₆ being as defined above.

Among the compounds of general formula (I) which are subject-matters of the invention, a seventh group of compounds is composed of the compounds for which;
R represents a hydrogen atom or a methyl, ethyl, propyl, isobutyl or allyl group, the methyl, ethyl or isobutyl group or groups being optionally substituted by one or more groups chosen, independently of one another, from the fluorine atom or hydroxyl, cyclopropyl or phenyl groups;
R₁ represents a phenyl or naphthyl group optionally substituted by one or more halogen atoms or methyl, methoxy, trifluoromethyl. NH₂ or hydroxyl groups;
R₂ represents one or more substituents chosen from the hydrogen atom, halogen atoms or methyl, ethyl. NH₂, methoxy, trifluoromethyl, methanesulphanyl or ethanesulphonyl groups;
   and their addition salts with an acid.

The combinations of the groups one to seven below as defined above also come within the scope of the invention.

Mention may in particular be made, among the compounds of general formula (I) which are subject-matters of the invention, of the following compounds;
N-[(4-Aminophenyl)(2-azabicyclo[2.1.1]hex-1-yl)methyl]-2-chloro-3-(trifluoromethyl)-benzamide and its hydrochloride;
N-[(2-Azabicyclo[2.1.1]hex-1-yl)(m-tolyl)methyl]-2-chloro-3-(trifluoromethyl)benzamide and its hydrochloride;
N-[(2-Azabicyclo[2.1.1]hex-1-yl)(3-methoxyphenyl)methyl]-2-chloro-3-(trifluoro-methyl)benzamide and its hydrochloride;
N-[(2-Azabicyclo[2.1.1]hex-1-yl)[3-(trifluoromethyl)phenyl]methyl]-2-chloro-3-(trifluoromethyl)benzamide and its hydrochloride;
N-[(2-Azabicyclo[2.1.1]hex-1-yl)(phenyl)methyl]-2-chloro-3-(trifluoromethyl)benzamide;
2-Amino-N-[(2-azabicyclo[2.1]hex-1-yl)(phenyl)methyl]-5-bromo-4-chlorobenzamide;
N-[(2-Azabicyclo[2.1.1]hex-1-yl)(phenyl)methyl]-2-methyl-3-(trifluoromethyl)-benzamide;
N-[(2-Azabicyclo[2.1.1]hex-1-yl)(phenyl)methyl]-2,6-dichloro-3-(trifluoromethyl)-benzamide;
N-[(2-Azabicyclo[2.1.1]hex-1-yl)(phenyl)methyl]-2-chloro-5-(trifluoromethyl)benzamide and its hydrochloride;
N-[(2-Azabicyclo[2.1.1]hex-1-yl)(phenyl)methyl]-2-chloro-6-fluoro-3-methylbenzamide;
N-[(2-Azabicyclo[2.1.1]hex-1-yl)(phenyl)methyl]-2-chloro-5-(methylsulphanyl)-benzamide;
N-[(2-Azabicyclo[2.1.1]hex-1-yl)(phenyl)methyl]-2-chloro-3-methylbenzamide and its hydrochloride;
(−)N-[(2-Azabicyclo[2.1.1]hex-1-yl)(phenyl)methyl]-2-chloro-3-(trifluoromethyl)-benzamide and its hydrochloride;
(+)-N-[(2-Azabicyclo[2.1.1]hex-1-yl)(phenyl)methyl]-2-chloro-3-(trifluoromethyl)-benzamide and its hydrochloride;
N-[(2-Azabicyclo[2.1.1]hex-1-yl)(phenyl)methyl]-4-chloro-5-(ethanesulphonyl)-2-methoxybenzamide and its hydrochloride;
N-[(2-Azabicyclo[2.1.1]hex-1-yl)(4-fluorophenyl)methyl]-2-chloro-3-(trifluoromethyl)-benzamide;
N-[(2-Azabicyclo[2.1.1]hex-1-yl)(naphth-2-yl)methyl]-2-chloro-3-(trifluoromethyl)-benzamide and its hydrochloride;
2-Chloro-N-{(phenyl)[2-methyl-2-azabicyclo[2.1.1]hex-1-yl]methyl}-3-(trifluoromethyl)-benzamide and its hydrochloride;
2-Chloro-N-[(2-ethyl-2-azabicyclo[2.1.1]hex-1-yl)(phenyl)methyl]-3-(trifluoromethyl)-benzamide and its hydrochloride;
N-[(2-Allyl-2-azabicyclo[2.1.1]hex-1-yl)(phenyl)methyl]-2-amino-5-bromo-4-chloro-benzamide;
2-Amino-5-bromo-4-chloro-N-[(phenyl)(2-propyl-2-azabicyclo[2.1.1]hex-1-yl)methyl]-benzamide and its hydrochloride;
2,6-Dichloro-N-{[2-(2-hydroxy-2-methylpropyl)-2-azabicyclo[2.1.1]hex-1-yl](phenyl)methyl}-3-(trifluoromethyl)benzamide and its hydrochloride;
2,6-Dichloro-N-{(phenyl)[2-(2,2,2-trifluoroethyl)-2-azabicyclo[2.1.1]hex-1-yl]methyl}-3-(trifluoromethyl)benzamide;
N-[(2-Azabicyclo[2.1.1]hex-1-yl)(3-hydroxyphenyl)methyl]-2-chloro-3-(trifluoromethyl)-benzamide and its hydrochloride;
2-Chloro-N-[(2-cyclopropylmethyl-2-azabicyclo[2.1.1]hex-1-yl)(phenyl)methyl]-3-(trifluoromethyl)benzamide and its hydrochloride;
(+)-2-Chloro-N-[(2-ethyl-2-azabicyclo[2.1.1]hex-1-yl)(phenyl)methyl]-3-(trifluoromethyl)benzamide and its hydrochloride;
N-[(2-Azabicyclo[2.1.1]hex-1-yl)(phenyl)methyl]-2,6-dimethylbenzamide and its hydrochloride;
N-[(2-Azabicyclo[2.1.1]hex-1-yl)(phenyl)methyl]-2-ethylbenzamide and its hydrochloride;
N-[(2-Azabicyclo[2.1.1]hex-1-yl)(m-tolyl)methyl]-2,6-dichloro-3-(trifluoromethyl)-benzamide and its hydrochloride;
N-[(2-Ethyl-2-azabicyclo[2.1.1]hex-1-yl)(phenyl)methyl]-2,6-dimethylbenzamide and its hydrochloride;
2-Ethyl-N-[(2-ethyl-2-azabicyclo[2.1.1]hex-1-yl)(phenyl)methyl]benzamide and its hydrochloride;
N-[(2-Azabicyclo[2.1.1]hex-1-yl)(phenyl)methyl]-6-chloro-2-methyl-3-(trifluoromethyl)-benzamide and its hydrochloride;
N-[(2-Benzyl-2-azabicyclo[2.1.1]hex-1-yl)(phenyl)methyl]-2-chloro-3-(trifluoromethyl)-benzamide and its hydrochloride.

The compounds of the invention exhibit a specific activity as inhibitors of glycine transporters GlyT1, in particular an improved activity profile and an improved safety profile.

The compounds of general formula (I) can be prepared by a process illustrated by the following Scheme 1.

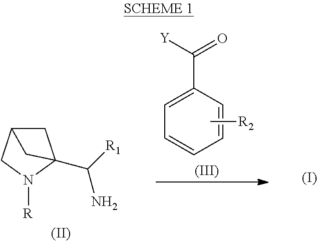

A diamine of general formula (II), in which R and R₁ are as defined above, in particular when R represents a hydrogen atom or an allyl or phenylmethyl group, is coupled with an activated acid, for example an acid activated via a mixed anhydride or an acid chloride, of general formula (III), in which Y represents a leaving group derived, for example, from benzotriazole or acylurea or a halogen atom and R₂ is as defined above, using methods known to a person skilled in the art.

The compounds of general formula (I) in which R represents a hydrogen atom can also be prepared from compounds of general formula (I) in which R represents:
either a phenylmethyl group, by deprotecting the nitrogen by hydrogenolysis,
or an alkenyl group, preferably an allyl group, by deprotecting the nitrogen, for example with a palladium(0) complex, according to methods known to a person skilled in the art.

The compounds of general formula (I) in which R is other than the hydrogen atom can also be prepared from compounds of general formula (I) in which R represents a hydrogen atom either by alkylation of the said compound of general formula (I) with a halide or mesylate of the RX type, in which R is as defined above and X is mesylate or halogen, in the presence of an inorganic base, for example potassium carbonate in acetonitrile; or by a reaction of Eschweiler-Clarke type or a reductive amination with an appropriate aldehyde or an appropriate ketone, according to methods known to a person skilled in the art; or with an appropriate epoxide derivative, according to methods known to a person skilled in the art.

The compounds of general formula (I) in which the R₁ group is a phenyl group substituted by a hydroxyl can be obtained from the corresponding compound of general formula (I) substituted by a methoxy, using methods known to a person skilled in the art.

The diamine of general formula (II) can be prepared by processes illustrated by the following Scheme 2, for the amine (IIa), and the following Scheme 3, for the amines (IIb) and (IIc):

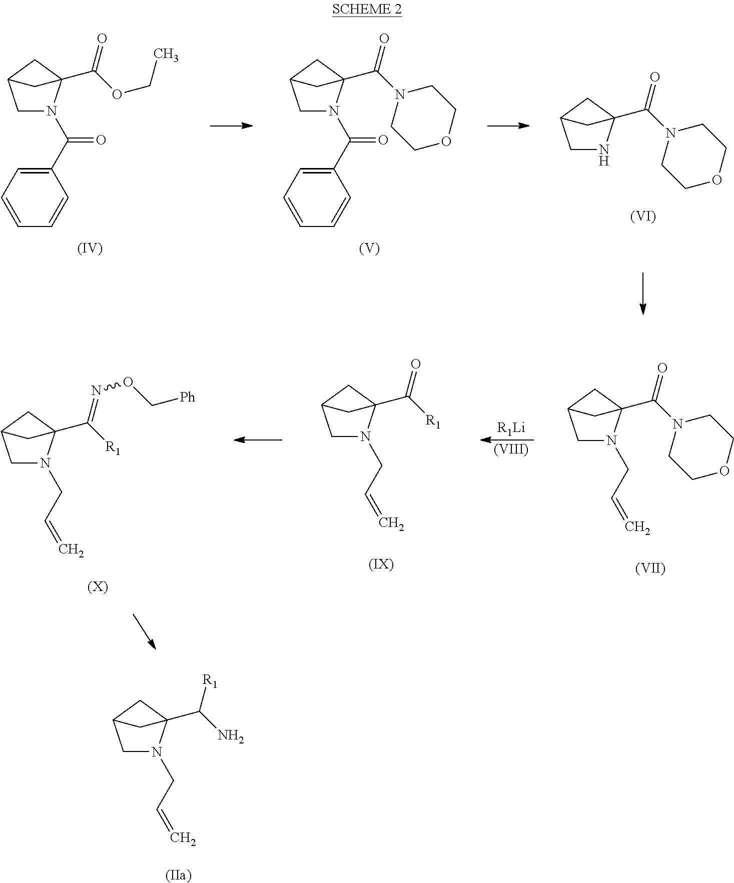

The ester (IV) is converted to the amide (V) by heating the trimethylaluminium complex and the appropriate amine, such as morpholine, at reflux of the solvent, such as toluene. The amine (V) can be deprotected, in order to obtain the compound (VI), by using a lithium compound of phenyllithium type in a solvent, such as tetrahydrofuran, at low temperature, for example at −70° C. An N-allylation is subsequently carried out using allyl bromide in the presence of a base, such as potassium carbonate, in a solvent, such as acetonitrile, at ambient temperature, in order to obtain the compound (VII). The morpholine amide of formula (VII) can be reacted with the lithiated aromatic compound of general formula (VIII), in which R₁ is as defined above, in an ethereal solvent, such as ether or tetrahydrofuran, at low temperature. A ketone of general formula (IX) is thus obtained and is reacted with O-benzylhydroxylamine hydrochloride, at reflux of pyridine, in order to obtain a Z/E mixture of oxime of general formula (X).

The oxime (X) is subsequently reduced at reflux of the ether by lithium aluminium hydride, in order to provide the diamine of formula (IIa).

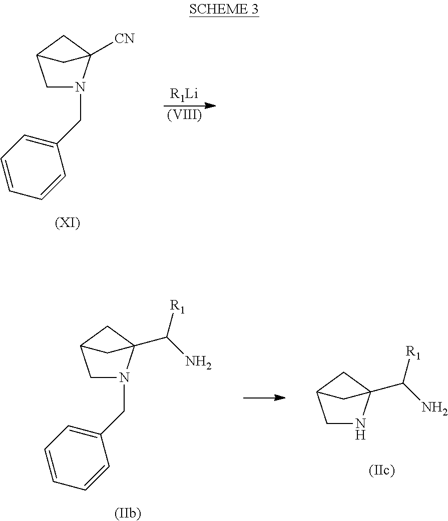

According to Scheme 3, a nitrile of formula (XI) is reacted with the lithiated aromatic compound of general formula (VIII), in which R₁ is as defined above, in an ethereal solvent, such as tetrahydrofuran or ether, at low temperature, for example −70° C. An imine is thus obtained and is reduced with a reducing agent, such as sodium borohydride, in a protic solvent, such as methanol, to give the amine of general formula (IIb). The amine (IIb) can be debenzylated by hydrogenation in the presence of palladium catalyst to provide the deprotected amine (IIc).

Furthermore, the chiral compounds of general formula (I) corresponding to the S or R enantiomers can be obtained by separation of the racemic compounds by high performance liquid chromatography (HPLC) on a chiral column or might be obtained by resolution of the racemic amine of general formula (II) by use of a chiral acid, such as dibenzoyltartaric acid, or by the fractional and preferential recrystallization of a diastereoisomeric salt.

The ester of formula (IV) is prepared according to a method described in J. Org. Chem., 2003, 9348-9355.

The nitrile of formula (XI) is prepared according to a method described in Tetrahedron: Asymmetry, 2006 (17), 252-258.

The lithiated derivatives of general formula (VIII) can be prepared according to methods known to a person skilled in the art.

The acids and acid chlorides of general formula (III) are available commercially or are prepared by analogy to methods known to a person skilled in the art.

The examples which will follow illustrate the preparation of some compounds of the invention. In these examples;
the elemental microanalyses, the IR and NMR spectra and chiral column HPLC confirm the structures and the enantiomeric purities of the compounds obtained,
for the NMR descriptions, “m” means multiplet, “s” singlet, “t” triplet, “d” doublet, “q” quartet, “d×d” double doublet, “t×t” triple triplet, “d×t” double triplet, and the like.
The numbers shown between brackets in the titles of the examples correspond to those in the 1^{st} column in Tables 1 and 2,
“decomp.” means “decomposition”,
the roman numerals in brackets correspond to the corresponding general formulae shown in the synthetic schemes,
the nomenclature employed is the nomenclature according to the IUPAC (International Union of Pure and Applied Chemistry) recommendations.

In the names of the compounds, the hyphen “-” forms part of the word and the “underline” symbol “_” is used only for the break at the line end; it is to be omitted in the absence of a break and should be replaced neither by an ordinary hyphen nor by a space.

### EXAMPLE 1

### Compound No. 9

N-[(2-Azabicyclo[2.1.1]hex-1-yl)(phenyl)methyl]-2-chloro-5-(trifluoromethyl)benzamide hydrochloride (1:1)

### 1.1 (2-Benzoyl-2-azabicyclo[2.1.1]hex-1-yl)(morpholin-4-yl)methanone (Compound of Formula V)

10 ml of morpholine (115 mmol) are added dropwise to a solution of 29 ml of 2N trimethylaluminum (58 mmol) in 200 ml of anhydrous toluene in a 500 ml three-necked flask under argon and the mixture is heated at 60° C. for 15 minutes. A solution of 20 g of ethyl 2-benzoyl-2-azabicyclo[2.1.1]hexane-1-carboxylate (77.1 mmol) in 190 ml of anhydrous toluene is transferred via a tube into the reaction medium, which is subsequently heated at reflux overnight. After cooling, the mixture is carefully hydrolysed with 60 ml of water while stirring. The precipitate formed is filtered off on Celite® and then rinsed with dichknronlethane. The filtrate is evaporated under reduced pressure.

The residue obtained is triturated from ether. 18.35 g of (2-benzoyl-2-azabicyclo[2.1.1]hex-1-yl)(morpholin-4-yl)methanone of general formula (V) are thus obtained in the form of a dark beige solid.

¹H NMR (400 MHz, d₆-DMSO) δ ppm 7.69 (d, J=8 Hz, 2H), 7.56-7.45 (m, 3H), 3.76 (d, J=7.7 Hz, 1H), 3.64-3.26 (m, 9H), 2.73 (t, J=2.7 Hz, 1H), 2.10 (m, 2H), 1.97 (m, 1H), 1.52 (m, 1H).

M.p.: 176-177° C.

### 1.2. (2-Azabicyclo[2.1.1]hex-1-yl)(morpholin-4-yl)methanone (Compound of Formula VI)

10 g of (2-benzoyl-2-azabicyclo[2.1.1]hex-1-yl)(morpholin-4-yl)methanone (V) (33.3 mmol) are placed in 400 ml of anhydrous tetrahydrofuran at −70° C. in a 1 l three-necked flask under argon. 50 ml of 0.8M phenyllithium (cyclohexane/ether) (40 mmol) are added dropwise and the solution obtained is left at −70° C. for 1 h.

Hydrolysis is carried out with 100 ml of water and the mixture is allowed to return to ambient temperature. After extracting, the organic phase is concentrated and then the residue is taken up in ether. This ethereal phase is poured into the preacidified aqueous phase. After extracting, the aqueous phase is basified with aqueous ammonia and than extracted with dichloromethane (3×200 ml). The organic phases are dried over sodium sulphate, filtered and evaporated under reduced pressure. 5.2 g of (2-azabicyclo[2.1.1]hex-1-yl)(morpholin-4-yl)methanone (VI) are thus obtained in the form of a dark beige solid.

¹H NMR (400 MHz, d₆-DMSO) δ ppm 3.71 (m, 2H), 3.55 (m, 4H), 3.44 (m, 2H), 2.87 (s, 2H), 2.69 (broad s, 1H), 2.60 (t, J=2.9 Hz, 1H), 1.84 (m, 2H), 1.43 (m, 2H).

M.p.: 97.5-98° C.

### 1.3. (2-Allyl-2-azabicyclo[2.1.1]hex-1-yl)(morpholin-4-yl)methanone (Compound of Formula VII)

7.4 g of (2-azabicyclo[2.1.1]hex-1-yl)(morpholin-4-yl)methanone (VI) (37.7 mmol) are placed in 100 ml of acetonitrile and 10.4 g of potassium carbonate (75.4 mmol) in a 500 ml round-bottomed flask. A solution of 3.9 ml of allyl bromide (45.2 mmol) is added dropwise to this suspension. The reaction medium is stirred overnight at ambient temperature and then concentrated under reduced pressure.

The residue is dissolved in 100 ml of dichloromethane. The organic phase is washed with water, dried over sodium sulphate, filtered and then evaporated under reduced pressure. 8.9 g of (2-allyl-2-azabicyclo[2.1.1]hex-1-yl)(morpholin-4-yl)methanone of general formula (VII) are thus obtained in the form of an oil.

¹H NMR (400 MHz, d₆-DMSO) δ ppm 5.85 (m, 1H), 5.24 (m, 1H), 5.09 (m, 1H), 378 (broad t, J=4.7 Hz, 2H), 3.54 (m, 4H), 3.44 (m, 2H), 3.05 (broad d, J=5.7 Hz, 2H), 2.69 (broad s, 2H), 2.56 (broad t, J=3 Hz, 1H), 1.83 (m, 2H), 1.68 (m, 2H).

### 1.4. (2-Allyl-2-azabicyclo[2.1.1]hex-1-yl)(phenyl)methanone (Compound of General Formula IX)

3.2 g of (2-allyl-2-azabicyclo[2.1.1]hex-1-yl)(morpholin-4-yl)methanone (VII) (13.5 mmol) are placed in 70 ml of tetrahydrofuran at −70° C. in a 250 ml three-necked flask under argon, 16.2 ml of 1M phenyllithium (cyclohexane/ether) are run in dropwise and the mixture is left at −70° C. for one hour. After hydrolysis with 20 ml of water, the mixture is allowed to return to ambient temperature. After evaporating the solvent under reduced pressure, the residue is taken up in ethyl acetate. After extracting, the organic phase is dried over sodium sulphate, filtered and evaporated under reduced pressure. The residue is purified by chromatography on a column of silica gel, elution being carried out with a mixture of petroleum ether and ethyl acetate. 2 g of (2-allyl-2-azabicyclo[2.1.1]hex-1-yl)(phenyl)methanone are thus obtained in the form of an oil (compound of general formula IX).

¹H NMR (400 MHz, d₆-DMSO) δ ppm 8.28 (m, 2H), 7.64 (txt, J=7.3 and 1.4 Hz, 1H), 7.52 (m, 2H), 5.73 (m, 1H), 5.20 (m, J=17 and 2 Hz, 1H), 5 (m, J=10 and 2 Hz, 1H), 2.99 (dxt, J=5.6 and 1.5 Hz, 2H), 2.86 (s, 2H), 2.70 (t, J=2.9 Hz, 1H), 1.99-1.85 (m, 4H).

### 1.5. (2-Allyl-2-azabicyclo[12.1.1]hex-1-4)(phenyl)methanone O-benzyloxime (Compound of General Formula X)

0.8 g of (2-allyl-2-azabicyclo[2.1.1]hex-1-yl)(phenyl)methanone (IX) (31 mmol) is placed in 12 ml of pyridine in a 50 ml round-bottomed flask and then 0.91 g of O-benzylhydroxylamine hydrochloride (7.4 mmol) is added. The reaction medium is heated at reflux overnight and then concentrated under reduced pressure.

The residue is taken up in water basified with aqueous ammonia and then extracted three times with dichloromethane. The organic phases are combined, washed with a saturated sodium chloride solution, dried over sodium sulphate, filtered and evaporated under reduced pressure. The crude product is purified by chromatography on a column of silica gel, elution being carried out with a mixture of dichloromethane and ammoniacal methanol. 1.2 g of (2-allyl-2-azabicyclo[2.1.1]hex-1-yl)(phenyl)methanone O-benzyloxime of general formula (X) are thus obtained in the form of an oil.

¹H NMR (400 MHz, d₆-DMSO) δ ppm 7.49-7.45 (m, 2H), 7.42-7.26 (m, 8H), 5.76 (m, 1H), 5.17 (m, J=17 Hz and 1.7 Hz, 1H), 5.09 (s, 1H), 5.03 (m, 1H), 3.06 (dxt, J=5.9 Hz and 1.4 Hz, 2H), 2.66 (broad s, 2H), 2.62 (broad t, J=3 Hz, 2H), 1.79 (m, 2H), 1.63 (m, 2H).

### 1.6. [(2-Allyl-2-azabicyclo[2.1.1]hex-1-yl)(phenyl)methyl]amine (IIa)

0.32 g of lithium aluminium hydride (8.4 mmol) is placed in 15 ml of ether in a 50 ml three-necked flask under nitrogen. A solution of 0.7 g of (2-allyl-2-azabicyclo[2.1.1]hex-1-yl)(phenyl)methanone O-benzyloxime (X) (2.1 mmol) in 3 ml of ether is subsequently added and then the mixture is heated at 40° C. for 3 hours. After cooling, the reaction medium is hydrolysed at 0° C. with 1.4 ml of a 0.1M aqueous double tartrate solution overnight.

After filtering the reaction medium, the filtrate is concentrated under reduced pressure. The residue is purified by chromatography on a column of silica gel, elution being carried out with a mixture of dichloromethane and ammoniacal methanol. 0.3 g of [(2-allyl-2-azabicyclo[2.1.1]hex-1-yl)(phenyl)methyl]amine (IIa) is thus obtained in the form of an oil.

¹H NMR (400 MHz, d₆-DMSO) δ ppm 7.36-7.15 (m, 5H), 5.87 (m, 1H), 5.23 (m, 1H), 5.06 (m, 1H), 4.14 (s, 1H), 3.36 (m, J=13.5 and 5.5 Hz, 1H), 3.06 (m, J=13.5 and 6.4 Hz, 1H), 2.76 (broad d, J=8 Hz, 1H), 2.43 (m, 2H), 1.78 (broad s, 2H), 1.39-1.21 (m, 3H), 1.08 (m, 1H).

### 1.7. N-[(2-Allyl-2-azabicyclo[2.1.1]hex-1-yl)(phenyl)methyl]-2-chloro-5-trifluoro-methyl)benzamide (Ia)

0.15 g of [(2-allyl-2-azabicyclo[2.1.1]hex-1-yl)(phenyl)methyl]amine (IIa) (0.66 mmol) is placed in 3 ml of dichloromethane at 0° C. in the presence of 0.18 g of potassium carbonate (1.31 mmol) in a 25 ml round-bottomed flask. A solution of 0.19 g of 2-chloro-5-(trifluoromethyl)benzoyl chloride (0.79 mmol) in 2 ml of dichloromethane is added and the mixture is left stirring at ambient temperature overnight. The reaction medium is subsequently diluted with 10 ml of dichloromethane and then washed successively with water (5 ml), a 1N sodium hydroxide solution (5 ml) and a saturated sodium chloride solution (5 ml).

The organic phase is dried over sodium sulfate, filtered and evaporated under reduced pressure. The residue is purified by chromatography on a column of silica gel, elution being carried out with a mixture of dichloromethane and ammoniacal methanol. 0.24 g of N-[(2-allyl-2-azabicyclo[2.1.1]hex-1-yl)(phenyl)methyl]-2-chloro-5-(trifluoromethyl)benzamide (Ia) is thus obtained in the form of an oil.

¹H NMR (400 MHz, d₆-DMSO) δ ppm 9.10 (d, J=8.8 Hz, 1H), 7.85 (d×d, J=8.5 and 2.3 Hz, 1H), 7.78 (d, J=8.5 Hz, 1H), 7.64 (d×d, J=2.2 Hz, 1H), 7.43-7.25 (m, 5H), 5.86 (m, 1H), 5.36 (d, J=8.7 Hz, 1H), 5.27 (m, 1H), 5.10 (m, 1H), 3.40-3.27 (m, 3H), 3.19 (m, 1H), 2.79 (m, J=8.4 Hz, 1H), 1.53 (m, 1H), 1.45-1.29 (m, 3H).

### 1.8. N-[(2-Azabicyclo[2.1.1]hex-1-yl)(phenyl)methyl]-2-chloro-5-(trifluoromethyl)benz-amide hydrochloride (1:1)

3.5 mg of palladiumtetrakis(triphenylphosphine) (0.003 mmol) and 0.14 g of N,N-dimethylbarbituric acid (0.9 mmol) in solution in 1 ml of dichloromethane are placed in a 10 ml round-bottomed flask under argon provided with a reflux condenser. The reaction medium is heated at 40° C. before adding 0.13 g of N-[(2-allyl-2-azabicyclo[2.1.1]hex-1-yl)(phenyl)methyl]-2-chloro-5-(trifluoromethyl)benzamide (Ia) (0.3 mmol) in 2 ml of dichloromethane and then the mixture is heated at 40° C. for a further 2 hours. After cooling, the mixture is diluted with 10 ml of dichloromethane and subsequently hydrolyzed with 5 ml of a sodium carbonate solution.

The organic phase is separated and washed twice with 5 ml of 1N hydrochloric acid. The aqueous phases are combined, then basified with aqueous ammonia to pH 9 and subsequently extracted twice with 25 ml of dichloromethane. The organic phases are dried over sodium sulphate, filtered and evaporated under reduced pressure. 0.1 g of N-[(2-azabicyclo[2.1.1]hex-1-yl)(phenyl)methyl]-2-chloro-5-(trifluoromethyl)benzamide is thus obtained, which product is salified in the hydrochloride form by dissolution of the base in ether, followed by addition of an excess of 1N hydrochloric acid in ether. The solid obtained is filtered off and then dried under vacuum.

¹H NMR (400 MHz, d₆-DMSO) δ ppm 9.48 (d, J=8.8 Hz, 1H), 9.13 (m, 1H), 8 (d, J=2.1 Hz, 1H), 7.88 (d×d, J=8.6 and 2.3 Hz, 1H), 7.78 (d, J=8.6 Hz, 1H), 7.50-7.35 (m, 5H), 5.69 (d, J=8.8 Hz, 1H), 3.41-3.19 (m, 2H), 2.79 (1, J=3 Hz, 1H), 2.03 (m, 1H), 1.79 (m, 1H), 1.55 (m, 2H).

M.p.=162.5-163.5° C.

### EXAMPLE 2

### Compound No. 5

N-[(2-azabicyclo[2.1.1]hex-1-yl)(phenyl)methyl]-2-chloro-3-(trifluoromethyl)benzamide hydrochloride (1:1).

### 2.1 [(2-Benzyl-2-azabicyclo[2.1.1]hex-1-yl)(phenyl)methyl]amine of General Formula (IIb)

3 g of 2-benzyl-2-azabicyclo[2.1.1]hexane-1-carbonitrile (XI) (15.1 mmol) are placed at −70° C. in 100 ml of anhydrous tetrahydrofuran in a 500 ml three-necked flask under argon, 37.8 ml of a 0.8M solution (cyclohexane/ether) of phenyllithium (30.2 mmol) are added dropwise.

The reaction mixture is left at −70° C. for two and a half hours and is then hydrolysed at −20° C. with 30 ml of water.

After extracting, the organic phase is concentrated and then the residue is taken up in 40 ml of methanol. 2.8 g of sodium borohydride (75 mmol) are added thereto portionwise. The reaction medium is left stirring at ambient temperature overnight.

After evaporating under reduced pressure, the residue is taken up in 100 ml of ether and 100 ml of water.

The medium is acidified with a 1N hydrochloric acid solution and then the ethereal phase is extracted.

The aqueous phase is basified with aqueous ammonia and then reextracted twice with 100 ml of dichloromethane. The organic phases are combined and then dried over sodium sulphate, filtered and evaporated under reduced pressure. 4.15 g of [(2-benzyl-2-azabicyclo[2.1.1]hex-1-yl)(phenyl)methyl]amine (IIb) are thus obtained in the form of an oil which crystallizes in the cold.

¹H NMR (200 MHz, CDCl₃) δ ppm 7.6-7.3 (m, 5H), 4.4 (s, 1H), 4.2 (d, J=16 Hz, 1H), 3.6 (d, J=16 Hz, 1H), 3.0 (d, J=9 Hz, 1H), 2.6 (m, 1H), 2.4 (d, J=9 Hz, 1H), 1.8 (broad s, 2H), 1.6-1.2 (m, 4H).

M.p.=63.5-64° C.

An analytical sample is obtained in the form of the hydrochloride by dissolution of the base in ether, addition of an excess of 1N hydrochloric acid in ether and then concentration under reduced pressure.

M.p.=140-142° C.

### 2.2 [(2-Azabicyclo[2.1.1]hex-1-yl)(phenyl)methyl]amine (IIc)

0.43 g of [(2-benzyl-2-azabicyclo[2.1.1]hex-1-yl)(phenyl)methyl]amine (IIb) (1.54 mmol) is placed in 20 ml of ethanol and 5 ml of 1N hydrochloric acid, in the presence of a spatula tip of 10% palladium-on-charcoal, in a Parr bottle under 4 atmospheres of hydrogen at 40° C. for 3 hours.

After filtering of the catalyst and then concentrating under reduced pressure, the residue is taken up in 30 ml of dichloromethane and 30 ml of water basified with aqueous ammonia. After extracting, the organic phase is dried over sodium sulphate, filtered and evaporated under reduced pressure. 0.24 g of [(2-azabicyclo[2.1.1]hex-1-yl)(phenyl)methyl]amine (IIc) is thus obtained in the form of a yellow oil which solidifies in the cold and which is used as is in the following stage.

M.p.=46.5-47° C.

An analytical sample is obtained in the form of the hydrochloride by dissolution of the base in ether, addition of an excess of 1N hydrochloric acid in ether and than concentration under reduced pressure.

¹H NMR (400 MHz, d₆-DMSO) δ ppm 10.12-8.71 (m, 4H), 7.46-7.35 (m, 6H), 4.83 (m, 1H), 3.15 (m, 2H), 2.72 (m, 1H), 2.10 (m, 1H), 1.89 (m, 1H), 1.57 (broad t, J=9.3 Hz, 1H), 1.36 (broad t, J=9.3 Hz, 1H).

M.p.=220-223° C. (decomp.)

2.3 N-[(2-azabicyclo[2.1.1]hex-1-yl)(phenyl)methyl]-2-chloro-3-(trifluoromethyl)benz-amide hydrochloride (1:1).

2.4 g of 2-chloro-3-(trifluoromethyl)benzoic acid (10.8 mmol), 1.45 g of hydroxybenzotriazole (10.8 mmol) and 2.1 g of 1-[3-(dimethylamino)propyl]-3-ethylcarbodiimide hydrochloride (10.8 mmol) are placed in solution in 20 ml of dichloromethane in a 250 ml round-bottomed flask and the mixture is stirred at ambient temperature for 15 minutes. 17 g (9.0 mmol) of [(2-azabicyclo[2.1.1]hex-1-yl)(phenyl)methyl]amine (IIc) in solution in 20 ml of dichloromethane are added and the mixture is stirred at ambient temperature overnight.

The reaction medium is subsequently diluted with 10 ml of dichloromethane and then successively washed with water (5 ml), 1N sodium hydroxide solution (5 ml) and a saturated sodium chloride solution (5 ml).

The organic phase is dried over sodium sulphate, filtered and evaporated under reduced pressure.

1.8 g of N-[(2-azabicyclo[2.1.1]hex-1-yl)(phenyl)methyl]-2-chloro-3-(trifluoro-methyl)benzamide are thus obtained. An analytical sample is obtained in the form of the hydrochloride by dissolution of the base in dichloromethane, addition of an excess of 1N hydrochloric acid in ether and then concentration under reduced pressure.

¹H NMR (400 MHz, d₆-DMSO) δ ppm 9.09 (d, J=9 Hz, 1H), 7.94 (d×d, J=7.8 Hz and 1.8 Hz, 1H), 7.68 (m, 1H), 7.63 (m, 1H), 7.41-7.31 (m, 4H), 7.27 (m, 1H), 5.33 (d, J=8.8 Hz, 1H), 2.78 (m, 2H), 2.64 (t, J=2.9 Hz, 1H), 2.20 (m, 1H), 1.68 (m, 2H), 1.14 (m, 2H).

M.p.=148-150° C.

### EXAMPLE 3

### Compound No. 19

2-Chloro-N-[(2-ethyl-2-azabicyclo[2.1.1]hex-1-yl)(phenyl)methyl]-3-(trifluoromethyl)benzamide hydrochloride (1:1)

0.15 g of N-[(2-azabicyclo[2.1.1]hex-1-yl)(phenyl)methyl]-2-chloro-3-(trifluoro-methyl)benzamide (0.38 mmol) and 0.10 g of potassium carbonate (0.76 mmol) are placed in 2 ml of acetonitrile in a 25 ml round-bottomed flask and 40 μl of iodoethane (0.46 mmol) are added thereto.

The reaction medium is stirred at ambient temperature overnight and then concentrated under reduced pressure. The residue is subsequently diluted with 10 ml of dichloromethane and then washed with water (5 ml).

The organic phase is dried over sodium sulphate, filtered and evaporated under reduced pressure. 82 mg of 2-chloro-N-[(2-ethyl-2-azabicyclo[2.1.1]hex-1-yl)(phenyl)methyl]-3-(trifluoromethyl)benzamide are thus obtained, which product is salified in the form of the hydrochloride by dissolution of the base in dichloromethane, addition of an excess of 1N hydrochloric acid in ether and then concentration under reduced pressure.

¹H NMR (400 MHz, d₆-DMSO) δ ppm 9.06 (d, J=8.6 Hz, 1H), 7.94 (d×d, J=7.3 Hz and 2.1 Hz, 1H), 7.67-7.59 (m, 2H), 7.37 (m, 4H), 7.28 (m, 1H), 5.33 (d, J=8.8 Hz, 1H), 2.76-2.52 (m, 5H), 1.50 (m, 1H), 1.37 (m, 3H), 1.04 (t, J=7.2 Hz, 3H).

M.p.=152-155° C.

### EXAMPLE 4

### Compound No. 22

2,6-Dichloro-N-{[(2-(2-hydroxy-2-methylpropyl)-2-azabicyclo[2.1.1]hex-1-yl](phenyl)methyl}-3-(trifluoromethyl)benzamide hydrochloride (1:1)

196 mg of N-[(2-azabicyclo[2.1.1]hex-1-yl)(phenyl)methyl]-2,6-dichloro-3-(trifluoromethyl)benzamide (0.46 mmol) are placed in 2 ml of absolute ethanol in the presence of 0.81 ml of 2,2-dimethyloxirane (9.13 mmol) in a sealed tube under argon. The reaction medium is heated at 100° C. for 40 minutes using microwave radiation. After evaporating the solvent under reduced pressure, the residue is taken up in a mixture of water and dichloromethane. After extracting, the organic phase is dried over sodium sulphate, filtered and evaporated under reduced pressure. The residue is purified by chromatography on a column of silica gel, elution being carried out with a mixture of dichloromethane and methanol. 58 mg of 2,6-dichloro-N-{[2-(2-hydroxy-2-methylpropyl)-2-azabicyclo[2.1.1]hex-1-yl](phenyl)methyl}-3-(trifluoromethyl)-benzamide are thus obtained in the form of an oil, which product is salified by dissolution in ether, addition of an excess of 1N hydrochloric acid in ether and then concentration under reduced pressure.

¹H NMR (400 MHz, d₆-DMSO) δ ppm 9.91-9.46 (m, 2H), 7.97 (m, 1H), 779 (m, 1H), 7.60-7.29 (m, 5H), 5.87-5.54 (m, 1H), 5.31 (m, 1H), 3.93-3.21 (m, 4H), 2.75 (m, 1H), 2.25-1.43 (m, 4H), 1.31 (m, 6H).

M.p.: 178.5-179.0° C.

### EXAMPLE 5

### Compound No. 23

2,6-Dichloro-N-{(phenyl)[2-(2,2,2-trifluoroethyl)-2-azabicyclo[2.1.1]hex-1-yl]methyl}-3-(trifluoromethyl)benzamide

117 mg of N-[(2-azabicyclo[2.1.1]hex-1-yl)(phenyl)methyl]-2,6-dichloro-3-(trifluoromethyl)benzamide (0.27 mmol) are placed in 1.5 ml of absolute ethanol in the presence of 46 mg of sodium hydrogencarbonate (0.55 mmol) and 64 mg of 2,2,2-trifluoroethyl trifluoromethanesulphonate (0.27 mmol) in a sealed tube under argon. The reaction medium is heated at 100° C. for 4 h. After evaporating the solvent under reduced pressure, the residue is taken up in water and dichloromethane. After extracting, the organic phase is dried over sodium sulphate, filtered and evaporated under reduced pressure. The residue is purified by chromatography on a column of silica gel, elution being carried out with a mixture of dichloromethane and methanol, 58 mg of 2,6-dichloro-N-{(phenyl)[2-(2,2,2-trifluoroethyl)-2-azabicyclo[2.1.1]hex-1-yl]methyl}-3-(trifluoromethyl)benzamide are thus obtained.

¹H NMR (400 MHz, d₆-DMSO) δ ppm 7.59 (d, J=8.5 Hz, 1H), 7.40-7.17 (m, 5H), 6.62 (m, 1H), 5.08 (d, J=5 Hz, 1H), 3.26 (m, 1H), 3.07 (d, J=8.8 Hz, 1H), 2.92 (m, 1H), 2.72 (d, J=8.8 Hz, 2H), 2.57 (m, 1H), 155-1.12 (m, 4H).

M.p.: 82-83° C.

### EXAMPLE 6

### Compound No. 18

2-Chloro-N-[(2-methyl-2-azabicyclo[2.1.1]hex-1-yl)(phenyl)methyl]-3-(trifluoromethyl)benzamide hydrochloride (1:1).

0.15 g of N-[(2-azabicyclo[2.1.1]hex-1-yl)(phenyl)methyl]-2-chloro-(3-trifluoromethyl)-benzamide (0.39 mmol) and 2 ml of formaldehyde are placed in 2 ml of formic acid in a 25 ml round-bottomed flask. The reaction mixture is heated at 100° C. overnight. After cooling, the medium is hydrolysed, basified to pH=9 with aqueous ammonia and then extracted with ethyl acetate. The organic phase is dried over sodium sulphate, filtered and evaporated under reduced pressure. 2,6-Dichloro-N-[(2-methyl-2-azabicyclo[2.1.1]hex-1-yl)(phenyl)methyl]-3-(trifluoromethyl)benzamide is obtained in the form of an oil, which product is salified by dissolution of the base in ether, addition of an excess of 1N hydrochloric acid in ether and then concentration under reduced pressure. 80 mg of 2,6-dichloro-N-[(2-methyl-2-azabicyclo[2.1.1]hex-1-yl)(phenyl)methyl]-3-(trifluoromethyl)benzamide hydrochloride are obtained.

¹H NMR (400 MHz, d₆-DMSO) δ ppm 10.80-10.42 (m, 1H), 9.51 (m, 1H), 8.31-7.29 (m, 8H), 5.78 (m, 1H), 4.01-1.03 (m, 10H).

M.p.: 168.5-169.5° C.

The other compounds described in Table 1 are obtained according to the methods described in Examples 1 to 7 starting from the appropriate amines of formula (IIa), (IIb) or (IIc), from appropriate lithium compounds of formula (VIII), from appropriate carboxylic acid derivatives of formula (III) or from appropriate alkylating agents.

The chemical structures of some compounds of the invention are illustrated in the following Table 1.

In the table:
In the “Salts” column,—denotes a compound in the form of the base, “HCl” denotes a hydrochloride and the FIGURE in brackets indicates the (base:acid) ratio,
in the R, R₁ and R₂ columns:
“Cl” means chlorine,
“Br” means bromine,
“CH₃” means methyl,
“C₂H₅” means ethyl,
“NH₂” means amino,
“OCH₃” means methoxy,
“Ph” means phenyl,
“SO₂C₂H₅” means ethanesulphonyl,
“CF₃” means trifluoromethyl,
in the “R₂” column, the FIGURE in front of the substituents indicates their position in the general formula (I),
the compounds in the table are provided in the form of the hydrochloride solvated by one or more water molecules,
compounds Nos. 13 and 14 in the table form a pair of enantiomers which are separated by preparative HPLC using a CHIRALpak® AD 20 μm column and, as solvent, a 95/5 isohexane/propan-2-ol mixture.

The physical properties, melting points and optical rotations of the compounds of Table 1 are given in Table 2.

In Table 2:
the [α_{D}]_{20° C.} column gives the analytical result for the optical rotation of the compounds in the table at the wavelength of 589 nM and at the temperature of 20° C. The solvent shown in brackets corresponds to the solvent employed in carrying out the measurement of the optical rotation in degrees and the letter “c” shows the concentration of the solvent in g/100 ml; “N.A.” means that the measurement of the optical rotation is not applicable,
the “LCMS MH⁺” column gives the molecular ion (M+H⁺) or (M⁺) observed by analysis of the products by mass spectrometry, either by LC-MS (Liquid Chromatography coupled to Mass Spectroscopy), carried out on a device of Agilent LC-MSD Trap type in positive ESI mode, or by direct introduction by MS (Mass Spectroscopy) on an Autospec M (EBE) device using the DCI-NH₃ technique or using the electron impact technique on a device of Waters GCT type.

TABLE 1
(I)

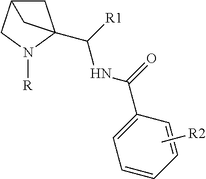

| | | | | | Stereo- |
|---|---|---|---|---|---|
| No. | R | R₁ | R₂ | Salts | chemistry |
| 1 | H | 4-NH₂—Ph | 2-Cl, 3-CF₃ | HCl (1:2) | racemic |
| 2 | H | 3-CH₃—Ph | 2-Cl, 3-CF₃ | HCl (1:1) | racemic |
| 3 | H | 3- | 2-Cl, 3-CF₃ | HCl (1:1) | racemic |
| | | OCH₃—Ph | | | |
| 4 | H | 3-CF₃—Ph | 2-Cl, 3-CF₃ | HCl (1:1) | racemic |
| 5 | H | Ph | 2-Cl, 3-CF₃ | HCl (1:1) | racemic |
| 6 | H | Ph | 2-NH₂, 4- | — | racemic |
| | | | Cl, 5-Br | | |
| 7 | H | Ph | 2-CH₃, | — | racemic |
| | | | 3-CF₃ | | |
| 8 | H | Ph | 2,6-(Cl)₂, | — | racemic |
| | | | 3-CF₃ | | |
| 9 | H | Ph | 2-Cl, | HCl (1:1) | racemic |
| | | | 5-CF₃ | | |
| 10 | H | Ph | 2-Cl, 3- | — | racemic |
| | | | CH₃, 6-F | | |
| 11 | H | Ph | 2-Cl, | — | racemic |
| | | | 5-SCH₃ | | |
| 12 | H | Ph | 2-Cl, 3-CH₃ | — | racemic |
| 13 | H | Ph | 2-Cl, 3-CF₃ | HCl (1:1) | chiral |
| | | | | | laevo- |
| | | | | | rotatory |
| 14 | H | Ph | 2-Cl, 3-CF₃ | HCl (1:1) | chiral |
| | | | | | dextro- |
| | | | | | rotatory |
| 15 | H | Ph | 2-OCH₃, | HCl (1:1) | racemic |
| | | | 4-Cl, 5- | | |
| | | | SO₂—C₂H₅ | | |
| 16 | H | 4-F—Ph | 2-Cl, 3-CF₃ | — | racemic |
| 17 | H | 2-naphthyl | 2-Cl, 3-CF₃ | HCl (1:1) | racemic |
| 18 | CH₃ | Ph | 2-Cl, 3-CF₃ | HCl (1:1) | racemic |
| 19 | C₂H₅ | Ph | 2-Cl, 3-CF₃ | HCl (1:1) | racemic |
| 20 | allyl | Ph | 2-NH₂, 4- | — | racemic |
| | | | Cl, 5-Br | | |
| 21 | n-C₃H₇ | Ph | 2-NH₂, 4- | HCl (1:1) | racemic |
| | | | Cl, 5-Br | | |
| 22 | CH₂—C(CH₃)₂OH | Ph | 2,6-(Cl)₂, | HCl (1:1) | racemic |
| | | | 3-CF₃ | | |
| 23 | CH₂CF₃ | Ph | 2-Cl, 3-CF₃ | — | racemic |
| 24 | H | 3-OH—Ph | 2-Cl, 3-CF₃ | HCl (1:1) | racemic |
| 25 | CH₂—c-Pr | Ph | 2-Cl, 3-CF₃ | HCl (1:1) | racemic |
| 26 | C₂H₅ | Ph | 2-Cl, 3-CF₃ | HCl (1:1) | chiral |
| | | | | | dextro- |
| | | | | | rotatory |
| 27 | H | Ph | 2,6-(CH₃)₂ | HCl (1:1) | racemic |
| 28 | H | Ph | 2-C₂H₅ | HCl (1:1) | racemic |
| 29 | H | 3-CH₃—Ph | 2,6-Cl₂, | HCl (1:1) | racemic |
| | | | 3-CF₃ | | |
| 30 | C₂H₅ | Ph | 2,6-(CH₃)₂ | HCl (1:1) | racemic |
| 31 | C₂H₅ | Ph | 2-C₂H₅ | HCl (1:1) | racemic |
| 32 | H | Ph | 2-CH₃, 3- | HCl (1:1) | racemic |
| | | | CF₃, 6-Cl | | |
| 33 | CH₂Ph | Ph | 2-Cl, 3-CF₃ | HCl (1:1) | racemic |

TABLE 2

| | | | LCMS |
|---|---|---|---|
| No. | M.p. (° C.) | [α_{D}]_{20° C.} ° | MH⁺ |
| 1 | 195-197 | N.A. | 410 |
| 2 | 159.5-160.5 | N.A. | 409 |
| 3 | 143-144 | N.A. | 425 |
| 4 | 214-217 | N.A. | 463 |
| 5 | 148-150 | N.A. | 395 |
| 6 | 89.5-90 | N.A. | 419/420 |
| 7 | 146-147 | N.A. | 375 |
| 8 | 80.5-81.5 | N.A. | 429 |
| 9 | 162.5-163.5 | N.A. | 395 |
| 10 | 123-124 | N.A. | 359 |
| 11 | 115-117 | N.A. | 373 |
| 12 | 139.5-140.5 | N.A. | 341 |
| 13 | 140-150 | −7.85 (CHCl₃) c = 0.40 g/100 ml | 395 |
| 14 | 160-170 | +6.24 (CHCl₃) c = 0.33 g/100 ml | 395 |
| 15 | 232-233 | N.A. | 449 |
| 16 | 172.5-173 | N.A. | 413 |
| 17 | 135-170 | N.A. | 445 |
| 18 | 168.5-169.5 | N.A. | 409 |
| 19 | 152-155 | N.A. | 423 |
| 20 | 66-67 | N.A. | 460 |
| 21 | 153-154 | N.A. | 462 |
| 22 | 178.5-179 | N.A. | 501 |
| 23 | 82-83 | N.A. | 511 |
| 24 | 187-188 | N.A. | 411 |
| 25 | 240.5-241.5 | N.A. | 449 |
| 26 | 257-259 | +22.64 (CHCl₃) c = 0.738 g/100 ml | 423 |
| 27 | 273.5-274.5 | N.A. | 321 |
| 28 | 264-265 | N.A. | 321 |
| 29 | 146-147 | N.A. | 443 |
| 30 | 212-214 | N.A. | 349 |
| 31 | 241-242 | N.A. | 349 |
| 32 | 200-201 | N.A. | 409 |
| 33 | 244-245.5 | N.A. | 485 |

The compounds of the invention have been subjected to a series of pharmacological trials which have demonstrated their advantage as substances possessing therapeutic activities.

### Study of Glycine Transportation in SK-N-MC Cells Expressing the Native Human Transporter GlyT1.

The uptake of [¹⁴C]glycine is studied in SK-N-MC cells (human neuroepithelial cells) expressing the native human transporter GlyT1 by measuring the radioactivity incorporated in the presence or absence of the test compound. The cells are cultured as a monolayer for 48 hours in plates pretreated with 0.02% fibronectin. On the day of the experiment, the culture medium is removed and the cells are washed with Krebs-HEPES (4-(2-hydroxyethyl)piperazine-1-ethanesulphonic acid) buffer at pH 7.4. After preincubation for 10 minutes at 37° C. in the presence either of buffer (control batch) or of test compound at various concentrations or of 10 mM of glycine (determination of the non-specific uptake), 10 μM of [¹⁴C]glycine (specific activity 112 mCi/mmol) are subsequently added. Incubation is continued for 10 min at 37° C. and the reaction is halted by washing twice with pH 7.4 Krebs-HEPES buffer. The radioactivity incorporated by the cells is then estimated after adding 100 μl of liquid scintillant and stirring for 1 h. Counting is carried out on a Microbeta Tri-Lux™ counter. The effectiveness of the compound is determined by the IC₅₀, the concentration of the compound which reduces by 50% the specific uptake of glycine, defined by the difference in radioactivity incorporated by the control batch and the batch which received the 10 mM glycine.

The compounds of the invention have, in this test, an IC₅₀ of the order of 0.001 to 10 μM.

Some examples of IC₅₀ results for compounds according to the invention are shown in Table 3.

TABLE 3

| Compound | IC₅₀ (μM) |
|---|---|
| 1 | 1.1 |
| 5 | 0.026 |
| 11 | 0.16 |
| 18 | 0.021 |
| 22 | 0.11 |
| 23 | 0.30 |
| 30 | 0.006 |
| 31 | 0.0016 |

The results of the trials carried out on the chiral compounds of the invention and their racemates in the general formula (I) in which R₂ represents in particular one or more halogen atoms or trifluoromethyl groups show that they are inhibitors of the glycine transporter GlyT1 present in the brain.

These results suggest that the compounds of the invention can be used for the treatment of cognitive and/or behavioural disorders associated with neurodegenerative diseases or dementia; for the treatment of psychoses, in particular schizophrenia (deficit form and productive form); or acute or chronic extrapyramidal symptoms induced by neuroleptics; for the treatment of various forms of anxiety, panic attacks, phobias or obsessive-compulsive disorders; for the treatment of various forms of depression, including pyschotic depression; for the treatment of bipolar disorders, manic disorders or mood disorders; or for the treatment of disorders due to alcohol abuse or withdrawal, disorders of sexual behaviour, eating disorders, migraine, pain or sleep disorders.

The compounds according to the invention can thus be used in the preparation of medicaments, in particular of medicaments which are inhibitors of the glycine transporter GlyT1.

Thus, according to another of its aspects, the subject-matter of the invention is medicaments which comprise a compound of formula (I) or an addition salt of the latter with a pharmaceutically acceptable acid or also a hydrate or a solvate of the compound of formula (I).

Another subject-matter of the present invention is pharmaceutical compositions comprising an effective dose of at least one compound according to the invention, in the form of the base or a pharmaceutically acceptable salt or solvate, as a mixture, if appropriate, with suitable excipients.

The said excipients are chosen according to the pharmaceutical form and the method of administration desired.

The pharmaceutical compositions according to the invention may thus be intended for oral, sublingual, subcutaneous, intramuscular, intravenous, topical, intratracheal, intranasal, transdermal, rectal or intraocular administration.

The unit administration forms can be, for example, tablets, gelatin capsules, granules, powders, solutions or suspensions to be taken orally or to be injected, patches or suppositories. Ointments, lotions and collyria can be envisaged for topical administration.

The said unit forms are dosed to allow a daily administration of 0.01 to 20 mg of active principle per kg of body weight, according to the pharmaceutical dosage form.

To prepare tablets, a pharmaceutical vehicle, which can be composed of diluents, such as, for example, lactose, microcrystalline cellulose or starch, and formulation adjuvants, such as binders (polyvinylpyrrolidone, hydroxypropylmethylcellulose, and the like), flow agents, such as silica, or lubricants, such as magnesium stearate, stearic acid, glyceryl tribehenate or sodium stearylfumarate, is added to the micronized or unmicronized active principle. Wetting or surface-active agents, such as sodium lauryl sulphate, can also be added. The preparation techniques can be direct tableting, dry granulation, wet granulation or hot melt.

The tablets can be bare, coated with sugar, for example with sucrose, or coated with various polymers or other appropriate materials. They can be designed to make possible rapid, delayed or sustained release of the active principle by virtue of polymer matrices or of specific polymers used in the coating.

To prepare gelatin capsules, the active principle is mixed with dry pharmaceutical vehicles (simple mixing, dry or wet granulation, or hot melt) or liquid or semisolid pharmaceutical vehicles.

The gelatin capsules can be hard or soft and coated or uncoated with a thin film, so as to have a rapid, sustained or delayed activity (for example, for an enteric form).

A composition in the form of a syrup or an elixir or for administration in the form of drops can comprise the active principle in conjunction with a sweetener, preferably a calorie-free sweetener, methylparaben or propylparaben, as antiseptic, a flavour enhancer and a colorant.

The water-dispersible powders and granules can comprise the active principle as a mixture with dispersing agents or wetting agents, or dispersing agents, such as polyvinylpyrrolidone, as well as with sweeteners and flavour-correcting agents.

Recourse is had, for rectal administration, to suppositories prepared with binders which melt at the rectal temperature, for example cocoa butter or polyethylene glycols.

Use is made, for parental administration, of aqueous suspensions, isotonic saline solutions or injectable sterile solutions comprising pharmacologically compatible dispersing agents and/or wetting agents, for example propylene glycol or butylene glycol.

The active principle can also be formulated in the form of microcapsules, optionally with one or more vehicles or additives or else with a polymer matrix or with a cyclodextrin (patches or sustained release forms).

The topical compositions according to the invention comprise a medium compatible with the skin. They can be provided in particular in the form of aqueous, alcoholic or aqueous/alcoholic solutions, of gels, of water-in-oil or oil-in-water emulsions having the appearance of a cream or of a gel, of microemulsions or of aerosols or in the form of vesicular dispersions comprising ionic and/or nonionic lipids. These pharmaceutical dosage forms are prepared according to methods conventional in the fields under consideration.

By way of example, a unit administration form of a compound according to the invention in the tablet form can comprise the following components:

| | |
|---|---|
| Compound according to the invention | 50.0 mg |
| Mannitol | 223.75 mg |
| Croscarmellose sodium | 6.0 mg |
| Maize starch | 15.0 mg |
| Hydroxypropylmethylcellulose | 2.25 mg |
| Magnesium stearate | 3.0 mg |

Orally, the dose of active principle administered daily can reach from 0.1 to 20 mg/kg, taken once or on several occasions.

There may be specific cases where higher or lower dosages are appropriate; such dosages do not depart from the scope of the invention. According to the usual practice, the dosage appropriate to each patient is determined by the physician according to the method of administration and the weight and the response of the said patient.

The present invention, according to another of its aspects, also relates to a method for the treatment of the pathologies indicated above which comprises the administration, to a patient, of an effective dose of a compound according to the invention or one of its pharmaceutically acceptable salts.

1. A compound of general formula (I): 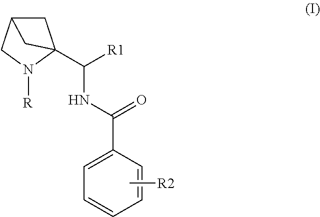
in which
R represents a hydrogen atom or a group chosen from (C₁-C₆)alkyl or (C₃-C₇)cycloalkyl groups, these groups optionally being substituted by one or more groups chosen, independently of one another, from the fluorine atom or (C₃-C₇)cycloalkyl, (C₂-C₄)alkenyl, phenyl, (C₁-C₆)alkoxy or hydroxyl groups; the phenyl group is optionally substituted by one or more (C₁-C₆)alkoxy groups;
R₁ represents a phenyl or naphthyl group which is optionally substituted by one or more substituents chosen, independently of one another, from halogen atoms or (C₁-C₆)alkyl, (C₁-C₆)alkoxy, halo(C₁-C₆)alkyl, NR₄R₅, NR₃C(O)OR₄, NR₃SO₂R₄, NR₃C(O)R₆, hydroxyl, halo(C₁-C₆)alkoxy, (C₁-C₆)alkylthio, (C₁-C₆)alkyl-SO₂, phenyl or heteroaryl groups, the phenyl group being optionally substituted by one or more substituents independently chosen from halogen atoms or (C₁-C₆)alkyl, (C₁-C₆)alkoxy, halo(C₁-C₆)alkyl, NR₄R₅, NR₃C(O)OR₄, NR₃SO₂R₄, NR₃C(O)R₆, hydroxyl, halo(C₁-C₆)alkoxy, (C₁-C₆)alkylthio or (C₁-C₆)alkyl-SO₂ groups and the heteroaryl group being optionally substituted by one or more substituents independently chosen from halogen atoms or (C₁-C₆)alkyl, (C₁-C₆)alkoxy, halo(C₁-C₆)alkyl or NR₄R₅ groups;
R₂ represents one or more substituents chosen from the hydrogen atom, halogen atoms or (C₁-C₆)alkyl, (C₃-C₇)cycloalkyl, (C₃-C₇)cycloalkyl(C₁-C₃)alkyl, halo(C₁-C₆)alkyl, (C₁-C₆)alkoxy, NR₄R₅, phenyl, heteroaryl, cyano, acetyl, (C₁-C₆)alkylthio, (C₁-C₆)alkylsulphonyl, carboxyl or (C₁-C₆)alkoxycarbonyl groups; the phenyl group being optionally substituted by one or more substituents independently chosen from halogen atoms or (C₁-C₆)alkyl, (C₁-C₆)alkoxy, halo(C₁-C₆)alkyl, NR₄R₅, NR₃C(O)OR₄, NR₃SO₂R₄, NR₃C(O)R₆, hydroxyl, halo(C₁-C₆)alkoxy, (C₁-C₆)alkylthio or (C₁-C₆)alkylSO₂ groups and the heteroaryl group being optionally substituted by one or more substituents independently chosen from halogen atoms or (C₁-C₆)alkyl, (C₁-C₆)alkoxy, halo(C₁-C₆)alkyl or NR₄R₅ groups;
R₃, R₄ and R₅ represent, independently of one another, a hydrogen atom or a (C₁-C₆)alkyl group;
R₆ represents a (C₁-C₆)alkyl group;
R₄ and R₅ can together form, with the nitrogen atom which carries them, a ring chosen from azetidine, pyrrolidine, piperidine, morpholine, thiomorpholine, piperazine or azepine rings optionally substituted by a (C₁-C₆)alkyl group;
R₃ and R₄ can together form, with the atoms which carry them, a 5- or 6-membered ring;
R₃ and R₆ can together form, with the atoms which carry them, a 5- or 6-membered ring;
in the form of the base or of an addition salt with an acid.

2. The compound according to claim 1, wherein R represents a hydrogen atom or a (C₁-C₆)alkyl group optionally substituted by one or more groups chosen, independently of one another, from the fluorine atom or (C₂-C₄)alkenyl, hydroxyl, (C₃-C₇)cycloalkyl or phenyl groups.

3. The compound according to claim 1, wherein R₁ represents a phenyl or naphthyl group optionally substituted by one or more halogen atoms or (C₁-C₆)alkyl, (C₁-C₆)alkoxy, halo(C₁-C₆)alkyl, NR₄R₅ or hydroxyl groups.

4. The compound according to claim 1, wherein R₂ represents one or more substituents chosen from the hydrogen atom, halogen atoms or NR₄R₅, (C₁-C₆)alkoxy, halo(C₁-C₆)alkyl, (C₁-C₆)alkylthio or (C₁-C₆)alkyl-SO₂ groups.

5. The compound according to claim 1, wherein:
R represents a hydrogen atom or a methyl, ethyl, propyl, isobutyl or allyl group, the methyl, ethyl or isobutyl group or groups being optionally substituted by one or more groups chosen, independently of one another, from the fluorine atom or hydroxyl, cyclopropyl or phenyl groups;
R₁ represents a phenyl or naphthyl group optionally substituted by one or more halogen atoms or methyl, methoxy, trifluoromethyl, NH₂ or hydroxyl groups;
R₂ represents one or more substituents chosen from the hydrogen atom, halogen atoms or methyl, ethyl, NH₂, methoxy, trifluoromethyl, methanesulphanyl or ethanesulphonyl groups;
and their addition salts with an acid.

6. The compound according to claim 1, wherein said compound is:
N-[(4-Aminophenyl)(2-azabicyclo[2.1.1]hex-1-yl)methyl]-2-chloro-3-(trifluoromethyl)-benzamide and its hydrochloride;
N-[(2-Azabicyclo[2.1.1]hex-1-yl)(m-tolyl)methyl]-2-chloro-3-(trifluoromethyl)benzamide and its hydrochloride;
N-[(2-Azabicyclo[2.1.1]hex-1-yl)(3-methoxyphenyl)methyl]-2-chloro-3-(trifluoro-methyl)benzamide and its hydrochloride;
N-[(2-Azabicyclo[2.1.1]hex-1-yl)[3-(trifluoromethyl)phenyl]methyl]-2-chloro-3-(trifluoromethyl)benzamide and its hydrochloride;
N-[(2-Azabicyclo[2.1.1]hex-1-yl)(phenyl)methyl]-2-chloro-3-(trifluoromethyl)benzamide;
2-Amino-N-[(2-azabicyclo[2.1.1]hex-1-yl)(phenyl)methyl]-5-bromo-4-chlorobenzamide;
N-[(2-Azabicyclo[2.1.1]hex-1-yl)(phenyl)methyl]-2-methyl-3-(trifluoromethyl)benzamide;
N-[(2-Azabicyclo[2.1.1]hex-1-yl)(phenyl)methyl]-2,6-dichloro-3-(trifluoromethyl)benzamide;
N-[(2-Azabicyclo[2.1.1]hex-1-yl)(phenyl)methyl]-2-chloro-5-(trifluoromethyl)benzamide and its hydrochloride;
N-[(2-Azabicyclo[2.1.1]hex-1-yl)(phenyl)methyl]-2-chloro-6-fluoro-3-methylbenzamide;
N-[(2-Azabicyclo[2.1.1]hex-1-yl)(phenyl)methyl]-2-chloro-5-(methylsulphanyl)benzamide;
N-[(2-Azabicyclo[2.1.1]hex-1-yl)(phenyl)methyl]-2-chloro-3-methylbenzamide and its hydrochloride;
(−)-N-[(2-Azabicyclo[2.1.1]hex-1-yl)(phenyl)methyl]-2-chloro-3-(trifluoromethyl)benzamide and its hydrochloride;
(+)-N-[(2-Azabicyclo[2.1.1]hex-1-yl)(phenyl)methyl]-2-chloro-3-(trifluoromethyl)benzamide and its hydrochloride;
N-[(2-Azabicyclo[2.1.1]hex-1-yl)(phenyl)methyl]-4-chloro-5-(ethanesulphonyl)-2-methoxybenzamide and its hydrochloride;
N-[(2-Azabicyclo[2.1.1]hex-1-yl)(4-fluorophenyl)methyl]-2-chloro-3-(trifluoromethyl)-benzamide;
N-[(2-Azabicyclo[2.1.1]hex-1-yl)(naphth-2-yl)methyl]-2-chloro-3-(trifluoromethyl)benzamide and its hydrochloride;
2-Chloro-N-{(phenyl)[2-methyl-2-azabicyclo[2.1.1]hex-1-yl]methyl}-3-(trifluoromethyl)-benzamide and its hydrochloride;
2-Chloro-N-[(2-ethyl-2-azabicyclo[2.1.1]hex-1-yl)(phenyl)methyl]-3-(trifluoromethyl)benzamide and its hydrochloride;
N-[(2-Allyl-2-azabicyclo[2.1.1]hex-1-yl)(phenyl)methyl]-2-amino-5-bromo-4-chlorobenzamide;
2-Amino-5-bromo-4-chloro-N-[(phenyl)(2-propyl-2-azabicyclo[2.1.1]hex-1-yl)methyl]-benzamide and its hydrochloride;
2,6-Dichloro-N-{[2-(2-hydroxy-2-methylpropyl)-2-azabicyclo[2.1.1]hex-1-yl](phenyl)methyl}-3-(trifluoromethyl)benzamide and its hydrochloride;
2,6-Dichloro-N-(phenyl)[2-(2,2,2-trifluoroethyl)-2-azabicyclo[2.1.1]hex-1-yl]methyl}-3-(trifluoromethyl)benzamide;
N-[(2-Azabicyclo[2.1.1]hex-1-yl)(3-hydroxyphenyl)methyl]-2-chloro-3-(trifluoromethyl)-benzamide and its hydrochloride;
2-Chloro-N-[(2-cyclopropylmethyl-2-azabicyclo[2.1.1]hex-1-yl)(phenyl)methyl]-3-(trifluoromethyl)benzamide and its hydrochloride;
(+)-2-Chloro-N-[(2-ethyl-2-azabicyclo[2.1.1]hex-1-yl)(phenyl)methyl]-3-(trifluoromethyl)-benzamide and its hydrochloride;
N-[(2-Azabicyclo[2.1.1]hex-1-yl)(phenyl)methyl]-2,6-dimethylbenzamide and its hydrochloride;
N-[(2-Azabicyclo[2.1.1]hex-1-yl)(phenyl)methyl]-2-ethylbenzamide and its hydrochloride;
N-[(2-Azabicyclo[2.1.1]hex-1-yl)(m-tolyl)methyl]-2,6-dichloro-3-(trifluoromethyl)benzamide and its hydrochloride;
N-[(2-Ethyl-2-azabicyclo[2.1.1]hex-1-yl)(phenyl)methyl]-2,6-dimethylbenzamide and its hydrochloride;
2-Ethyl-N-[(2-ethyl-2-azabicyclo[2.1.1]hex-1-yl)(phenyl)methyl]benzamide and its hydrochloride;
N-[(2-Azabicyclo[2.1.1]hex-1-yl)(phenyl)methyl]-6-chloro-2-methyl-3-(trifluoromethyl)-benzamide and its hydrochloride; or
N-[(2-Benzyl-2-azabicyclo[2.1.1]hex-1-yl)(phenyl)methyl]-2-chloro-3-(trifluoromethyl)-benzamide and its hydrochloride.

7. A process for preparing the compound according to claim 1, comprising reacting a compound of general formula (II): 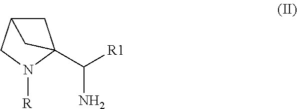
in which R and R₁ are as defined according to claim 1, with a compound of general formula (III): 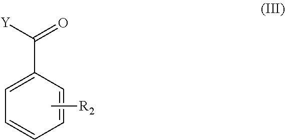
in which Y represents an activated OH group or a chlorine atom and R₂ is as defined according to claim 1.

8. A pharmaceutical composition comprising the compound of claim 1 or an addition salt of said compound with a pharmaceutically acceptable acid.

9. The pharmaceutical composition of claim 8 further comprising at least one pharmaceutically acceptable excipient.

10. A method of treating cognitive and/or behavioural disorders associated with neurodegenerative diseases or dementia in a patient in need thereof comprising administering to said patient a therapeutically effective amount of the pharmaceutical composition of claim 8.

11. A method of treating psychoses, schizophrenia (deficit form and productive form) or acute or chronic extrapyramidal symptoms induced by neuroleptics in a patient in need thereof comprising administering to said patient a therapeutically effective amount of the pharmaceutical composition of claim 8.

12. A method of treating various forms of anxiety, panic attacks, phobias or obsessive-compulsive disorders in a patient in need thereof comprising administering to said patient a therapeutically effective amount of the pharmaceutical composition of claim 8.

13. A method of treating depression, including psychotic depression; in the treatment of bipolar disorders, manic disorders or mood disorders; or in the treatment of disorders due to alcohol abuse or withdrawal, disorders of sexual behaviour, eating disorders or migraine in a patient in need thereof comprising administering to said patient a therapeutically effective amount of the pharmaceutical composition of claim 8.

14. A method of treating pain in a patient in need thereof comprising administering to said patient a therapeutically effective amount of the pharmaceutical composition of claim 8.

15. A method of treating sleep disorders in a patient in need thereof comprising administering to said patient a therapeutically effective amount of the pharmaceutical composition of claim 8.

16. (canceled)

17. (canceled)

18. (canceled)

19. (canceled)

20. (canceled)

21. (canceled)
